(19) United States
(12) Patent Application Publication
(10) Pub. No.: US 2023/0295584 A1
(43) Pub. Date: Sep. 21, 2023

(54) BIOSENSING ELEMENT BASED ON SPECIFIC BINDING OF CBM AND CELLULOSE

(71) Applicant: BIOLOGY INSTITUTE OF SHANDONG ACADEMY OF SCIENCES, Jinan (CN)

(72) Inventors: Weili GONG, Jinan (CN); Yaohong MA, Jinan (CN); Lei CAI, Jinan (CN); Binglian WANG, Jinan (CN); Qingjun MENG, Jinan (CN); Qingai LIU, Jinan (CN); Lan ZHENG, Jinan (CN); Yan YANG, Jinan (CN); Junhui YANG, Jinan (CN); Yiwei LI, Jinan (CN); Jianguo SHI, Jinan (CN); Qingye HAN, Jinan (CN)

(21) Appl. No.: 18/131,968

(22) Filed: Apr. 7, 2023

Related U.S. Application Data
(63) Continuation of application No. PCT/CN2021/116573, filed on Sep. 4, 2021.

(30) Foreign Application Priority Data
Oct. 9, 2020 (CN) 202011073250.X

(51) Int. Cl. C12N 9/04; C12N 9/42; G01N 33/00

(52) U.S. Cl. CPC C12N 9/0006; C12Y 101/03004; C12N 9/2437; G01N 33/0098

(57) ABSTRACT

Provided is a fusion protein for a biosensing element; the fusion protein includes glucose oxidase (GOD) and a carbohydrate-binding module (CBM); and the GOD is linked to the CBM by means of a linker peptide. The biosensing element specifically binds to cellulose based on CBM.

### REFERENCE TO SEQUENCE LISTING

The substitute sequence listing is submitted as a XML file filed via EFS-Web, with a file name of “Substitute_Sequence_Listing_SZOR-USP1231247.XML”, a creation date of Jul. 6, 2023, and a size of 19807 bytes. The substitute sequence Listing filed via EFS-Web is a part of the specifcation and is incorporated in its entirety by reference herein.

### TECHNICAL FIELD

The present disclosure relates to a biosensing element specifically binding to a nano cellulose carrier membrane prepared by using an oxidoreductase-linker-carbohydrate-binding module (CBM) fusion enzyme constructed by linking an oxidoreductase gene to a CBM gene by means of a linker peptide gene, belonging to the technical fields of biological enzyme genetic engineering and biosensing.

### BACKGROUND

As a key detection sensitive element in a biosensor, an oxidoreductase is effectively immobilized on the surface of a carrier and can maintain its activity state persistently and efficiently, and is an important guarantee for high sensitivity and high stability of biosensors. Immobilization of an enzyme molecule element on the surface of an electrode is achieved by randomly immobilizing an enzyme molecule on a carrier membrane with an area of less than 1 square centimetre by means of physical adsorption or covalent binding, or other manners. However, a biological active enzyme molecule has a spatial three-dimensional structure, its active center binding to a substrate has a specific orientation, interaction between the enzyme molecule and the carrier via non-specific physical adsorption or covalent binding often hides the active center of the enzyme molecule or makes it incorrectly orient, resulting in significant loss of enzyme activity and poor stability of a biosensor.

In recent years, immobilization of an enzyme using bioaffinity adsorption has attracted increasing attentions, its biggest advantages are that the enzyme molecule can specifically bind to a carrier material, the immobilization direction is controllable, and conformational change of the enzyme molecule is minimal. A carbohydrate-binding module (CBM) is a functional module that has no catalytic activity in a natural carbohydrate active enzyme molecule but can play an important role in recognizing and directionally binding to the substrate. There are many numbers of CBM, which has binding specificity to polysaccharides such as crystalline or non-crystalline celluloses, chitin and xylan. At present, about 100 thousands of CBM sequences are contained in a carbohydrate active enzyme database (http://www.cazy.org;), which belong to 86 families according to similarity of amino acid sequences (>30%), and 92 CBM structures are analyzed (http://www.rcsb.org/pdb/home/home/do). In the natural enzyme molecules, CBMs can be linked to the N end or C end of the catalysis module in a single module or a tendem module located in the same family or different families. With the development of molecular biology, synthetic biology, computer aided simulation and other technologies, the current researchers stimulate and analyze the overall and local molecular dynamics behaviors of proteins based on mining and analysis of biological big data such as sequence structures in combination with dynamics to establish a heterozygous library for accurate grafting of CBM and a target enzyme molecule, and are attempted to acquire a variety of CBM and enzyme molecule fusion proteins by utilizing a fusion DNA technology, so as to improve the affinity between the enzyme molecule and the substrate and the stability or enzyme activity of the enzyme molecule. Modification strategies for constructing a fusion enzyme by utilizing the specific affinity adsorption characteristic of CBM on cellulose and immobilizing the carrier with a nano cellulose membrane as a fusion enzyme molecule to improve the stability of an enzyme electrode sensor are currently almost not reported at home and abroad.

### SUMMARY

The present disclosure provides a fusion protein for a biosensing element, the fusion protein includes glucose oxidase and a carbohydrate-binding module (CBM).

In one embodiment, the glucose oxidase (GOD) is linked to the CBM by means of a linker peptide.

In one embodiment, the GOD is derived from Aspergillus niger An76, preferably, the amino acid sequence of the GOD is shown in SEQ ID NO.1, and the coding sequence of the GOD is shown in SEQ ID NO.2.

In one embodiment, the CBM is selected from second family CBM, preferably, the amino acid sequence of the CBM is shown in SEQ ID NO.3, and the coding sequence of the CBM is shown in SEQ ID NO.4.

The linker peptide is selected from (GGGGS)2, (EAAAK)3, or is selected from one or more of natural linker sequences linking a catalytic domain to a CBM2 domain in incision-β-xylanase (EM_PRO: Z81013.1) in a Thermobifida fusca genome; preferably, the amino acid sequence of the linker peptide is shown in SEQ ID NO.5, and the coding sequence of the linker peptide is shown in SEQ ID NO.6.

In a preferred implementation mode, the amino acid sequence of the fusion protein is shown in SEQ ID NO.7.

In another aspect, the present disclosure further provides a coding gene of the above fusion protein. The coding gene sequence of the fusion protein is shown in SEQ ID No. 8.

In still another aspect, the present disclosure further provides a method for preparing the above fusion protein, including steps of transforming the coding gene to Pichia pastoris followed by expressing and purifying.

In yet another aspect, the present disclosure further provides a biosensing element specifically binding to cellulose based on CBM, the biosensing element comprising the above fusion protein.

Further, the biosensing element further includes a cellulose membrane.

In also another aspect, the present disclosure further provides use of the above fusion protein in preparing a biosensing element.

In again another aspect, the present disclosure further provides a method for preparing a biosensing element specifically binding to cellulose based on CBM, the method comprising a step of contacting the above fusion protein with a cellulose membrane to prepare a fusion protein and cellulose membrane composite.

In a preferred implementation mode, in the fusion protein and cellulose membrane composite, the cellulose membrane serves as the immobilization carrier of the fusion protein. In a preferred embodiment, the above fusion protein solution is added onto the cellulose membrane and then dried to obtain the cellulose membrane immobilized with the fusion protein, and then the cellulose membrane immobilized with the fusion protein is fixed on the electrode, so as to the above biosensing element.

Sequence information is as follows:

| SEQ ID NO. | Description |
|---|---|
| 1 | GOD amino acid sequence |
| 2 | GOD nucleic acid sequence |
| 3 | CBM amino acid sequence |
| 4 | CBM nucleic acid sequence |
| 5 | Linker peptide amino acid sequence |
| 6 | Linker peptide nucleic acid sequence |
| 7 | GOD-NL-CBM2 amino acid sequence |
| 8 | GOD-NL-CBM2 nucleic acid sequence |

### BRIEF DESCRIPTION OF THE DRAWINGS

FIG. 1 is SDS-PAGE detection of heterologously expressed glucose oxidase; where (a) predicted wild type glucose oxidase (GOD) structure; (b) predicted fusion glucose oxidase (GOD-CBM2) structure; (c) strip M: protein standard, SDS-PAGE detection spectrum of 50 mM imidazole eluted GOD; (d) SDS-PAGE detection spectrum of 20 mM imidazole eluted GOD-NL-CBM2; (e) SDS-PAGE detection spectrum of concentrated GOD and GOD-NL-CBM2.

FIG. 2 is optimal temperature and optimal pH determination of GOD and GOD-NL-CBM2; (a) and (b) respectively show optimal temperature and optimal pH determination of GOD, GOD-NL-CBM2 and immobilized GOD-NL-CBM2; (c) and (d) respectively show SDS-PAGE detection of a protein content in supernatant after GOD reacts with a cellulose under different pH and temperature reaction systems; (e) and (f) respectively show SDS-PAGE detection of a protein content in GOD-NL-CBM2 and cellulose mixed supernatant under different pH and temperature reaction systems.

FIG. 3 is morphology and composition characteristic analysis of a cellulose membrane that does not react with or reacts with GOD; (a1), (a2) and (a3) respectively show scanning electron microscope (SEM), energy dispersive spectroscopy (EDS) and Fourier Transform Infrared Spectroscopy (FTIR) detection results of a cellulose membrane that does not react with GOD; (b1), (b2) and (b3) respectively show SEM, EDS and FTIR detection results of a cellulose membrane after reacting with GOD; (c1), (c2) and (c3) respectively show SEM, EDS and FTIR detection results of a cellulose membrane after reacting with GOD-NL-CBM2.

FIG. 4 is electrochemical behavior analysis of a GOD-NL-CBM2/cellulose membrane biological electrode; (a) a pattern diagram in which a cellulose membrane immobilized with GOD-NL-CBM2 is fixed on a platinum electrode (H₂O₂ electrode); (b) ESI detection results of nude electrode, cellulose membrane/electrode and GOD-NL-CBM2/cellulose membrane/electrode; (c) Cyclic voltammogram of GOD-NL-CBM2/cellulose membrane/electrode in PBS and 40 mM glucose solution.

FIG. 5 is results of current response of GOD-NL-CBM2/cellulose membrane/electrode to glucose; (a), (b), (c) and (d) respectively show that the concentrations of glucose are in ranges of 1.25 mM-5 mM, 2.5 mM-10 mM, 5.0 mM-20 mM and 10 mM-40 mM.

FIG. 6 is analysis on substrate selectivity, anti-interference ability and repeatability of GOD-NL-CBM2/cellulose membrane/electrode; (a) chronoamperometry I-T curve of GOD-NL-CBM2/cellulose membrane/electrode when reacting with different types of saccharides; (b) detection signal influence of a disruptor on GOD-NL-CBM2/cellulose membrane/electrode; (c) chronoamperometry I-T curve of 3 groups of GOD-NL-CBM2/cellulose membranes/electrodes when respectively reacting with different volumes of 40 mM glucoses added successively; (d) correlation coefficient (R²) analysis between an average current signal value and a glucose addition volume after 3 groups of GOD-NL-CBM2/cellulose membranes/electrodes respectively react with different volumes of 40 mM glucoses; (e) chronoamperometry I-T curves of 3 groups of GOD-NL-CBM2/cellulose membranes/electrodes when respectively reacting with different volumes of 5 mM glucoses added successively; (f) correlation coefficient (R²) analysis between an average current signal value and a glucose addition volume after 3 groups of GOD-NL-CBM2/cellulose membranes/electrodes respectively react with different volumes of 5 mM glucoses.

### DETAILED DESCRIPTION OF THE EMBODIMENTS

Next, the present disclosure will be further described in combination with embodiments. The followings are preferred embodiments of the present disclosure but are not intended to limit other forms of the present disclosure, any technical personnel familiar with this profession may change the technical contents disclosed above to equivalent embodiments. Any simple modifications or equivalent changes made to the following embodiments according to the technical essence of the present disclosure without departing from the contents of the technical solution of the present disclosure are all included within the scope of protection of the present disclosure.

### Example 1 Fusion Expression of CBM and GOD

CBM sequences specifically binding to a cellulose were selected from CBM1, CBM2, CBM3, CBM5 and CBM10 families, the amino acid frequency, functional architecture sequence spectrum and other information of different CBMs were subjected to statistic analysis by utilizing structural bioinformatics analysis tools (SWISS-MODEL, Clustal X, VMD and PyMOI software) to screen out CBM2 in Thermobifida fusca for construction of a hybrid enzyme.

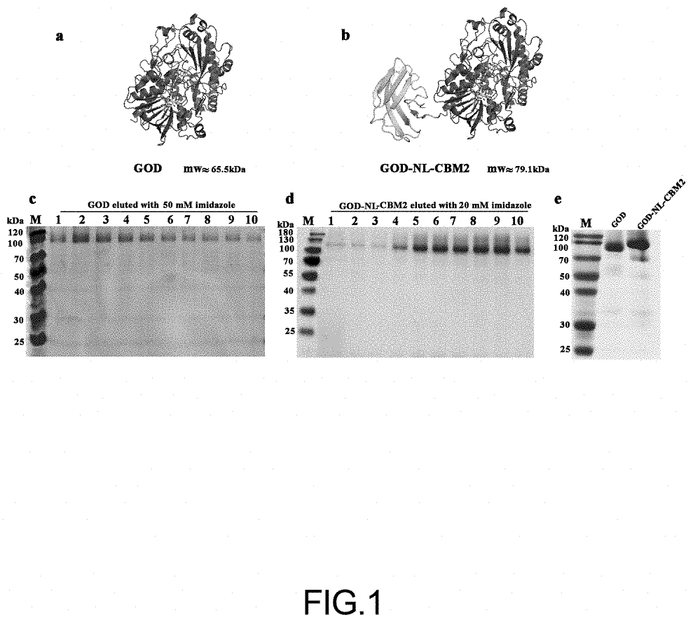

Based on structure characteristic analysis of CBM2 and GOD, fusion enzyme molecule linker sequences ((GGGGS)2 and (EAAAK)3) were selected from LinkerDB database. Meanwhile, a natural linker sequence (LGGDSSGGGPGEPGGPGGPGEPGGPGGPGEPGGPGDGT) linking a catalytic domain to a CBM2 domain in incision-β-xylanase (EM_PRO: Z81013.1) in a Thermobifida fusca genome; different fusion protein structures with or without linker connection were molded through an FPMOD tool and a fusion protein dynamic simulation system was constructed by using a Gromacs dynamic simulation tool, so as to analyze the root mean square of fluctuations, radius of gyration, hydrogen bonding, energy of protein secondary structure system and other parameters of the fusion enzyme, and then screen out a fusion enzyme molecule with a stable GOD-NL-CBM2 structure for subsequent heterologous expression. In the GOD-NL-CBM2, the linker peptide sequence of GOD and CBM2 is “LGGDSSGGGPGEPGGPGGPGEPGGPGGPGEPGGPGDGT”. The predicted GOD and GOD-NL-CBM2 structures are as shown in FIGS. 1a and 1b.

A primer was designed by using total cDNA of Aspergillus niger An 76 as a template for PCR to obtain a g9669.t1 gene encoding GOD, and a primer was designed by using plasmid pUC57-DNA as a template to obtain a gene encoding linker and CBM2. A GOD-NL-CBM2 fusion enzyme gene was obtained according to a Nanjing Novozan homologous recombinant kit method, the GOD-NL-CBM2 fusion enzyme gene achieved efficient expression of a GOD-NL-CBM2 fusion enzyme by using pPIC9K plasmid as an expression vector and using Pichia pastoris GS115 as an expression host.

(1) Total cDNA Extraction of Aspergillus niger an 76

Aspergillus niger An 76 strains were cultured for 36 h in a liquid culture medium (0.595% of NaNO₃, 0.0522% of KCl, 0.1497% of KH₂PO₄, 0.0493% of MgSO₄·7H₂O, 0.5% of yeast powder and 0.2% of tyrosine) at 30° C., 20 mL of bacterial fluid was taken to collect thalli, total RNA was extracted according to a RNA extraction kit method, and Aspergillus niger An 76 RNA was reversely transcripted into cDNA by using a reverse transcription kit method;

(2) Cloning of GOD-NL-CBM2 Fusion Enzyme Gene

Two pairs of primers P1, P2 and P3, P4 were designed according to the g9669.1 gene sequence and the pUC57-NL-CBM2 gene sequence in NCBI by utilizing a homologous recombination principle and sequences near Pichia pastoris GS115 cloning sites:

```
(SEQ ID NO. 9)
P1: 5′-gctgaagcttacgtagaattcctcccacactacatcaggag
              ca-3′
(SEQ ID No. 10)
P2: 5′-gaggtggtggtggtggtggtgc-3′
(SEQ ID No. 11)
P3: 5′-accaccaccaccaccacctcggcggcgactcctcc-3′
(SEQ ID No. 12)
P4: 5′-aaggcgaattaattcgcggccgctcagtggtggtggtggtg
              gt-3′
```

PCR amplification was performed by using An76 genome cDNA as a template and using P1 and P2 as primers to obtain a GOD gene, PCR amplification was performed by using a pUC57-NL-CBM2 gene as a template and using P3 and P4 as primers to obtain a linker peptide and a CBM2 gene. The PCR reaction was performed in a 50 μL system (referring to Phanata® Max Super-Fidelity DNA Polymerase), wherein reaction conditions were as follows: pre-denaturing for 3 min at 95° C. and then beginning to cycle, denaturing for 15 s at 95° C., annealing for 1 min at 60° C., and extending for 1 min at 72° C., after 30 cycles in total, extending at 72° C. for 5 min. The g9669.t1 gene and NL-CBM2 gene PCR fragments were respectively obtained by amplification and then recovered by cutting glue (purified with a product purification kit). Then, the g9669.t1 gene and NL-CBM2 gene PCR glue-cutting recovery fragments were linked to a Ppic9k plasmid vector by utilizing Nanjing Novozan ClonExxpress®Ultra One Step Cloning kit product, the reaction system is 10 μL (1 μL of linearized vector, 2 μL of g9669.t1 gene PCR fragment, 1.5 μL of NL-CBM2 gene PCR fragment, 5 μL of 2×ClonExpress Mix, 0.5 μL of ddH₂O), the above reaction system was evenly mixed by suction and then reacted for 15 min at 50° C., and then the reaction product was instantly cooled on ice so as to obtain a pPIC9k-GOD-NL-CBM2 fusion enzyme gene recombinant product.

(3) Transformation of Escherichia coli with pPIC9k-GOD-NL-CBM2 Fusion Enzyme Genes to Enrich Plasmids

A pPIC9k-GOD-NL-CBM2 fusion enzyme gene recombinant product was mixed with Escherichia coli DH5α, subjected to heat shock for 90 s, then coated onto a 100 μg/mL ampicillin-resistant LB agar culture plate, and cultured at 37° C. overnight. Single colonies were picked, and then plasmids were extracted for electrophoresis detection and stored at −20° C. A target fragment was detected by utilizing EcoRI and NotI enzyme digestion, subsequently a bacterial suspension was sequenced by the company, and Escherichia coli was transformed with the plasmids that were correctly sequenced by using the same method, so as to achieve plasmid enrichment.

(4) Transformation of Pichia pastoris Host Bacteria with pPIC9k-GOD-NL-CBM2

Pichia pastoris GS115 single colonies were inoculated into a test tube containing 5 mL of YPD liquid culture medium and cultured at 30° C. overnight. The above colonies were transferred into a baffled flask containing 50 mL of YPD liquid culture medium in an inoculation amount of 1% and then cultured at 30° C. overnight until OD600=1.3-1.5; the culture solution was centrifuged for 5 min at 4° C. at 1500 g, supernatant was discarded, and then cells were resuspended using 50 mL of ice bath double distilled water; the culture solution was centrifuged for 5 min at 4° C. at 1500 g, supernatant was discarded, and then cells were resuspended using 25 mL of ice bath double distilled water; the culture solution was centrifuged for 5 min at 4° C. at 1500 g, supernatant was discarded, and then cells were resuspended using 2 mL of 1 M sorbitol solution in ice bath; the culture solution was centrifuged for 5 min at 4° C. at 1500 g, supernatant was discarded, and then cells were resuspended using 100 μL of 1 M sorbitol solution in ice bath so that the volume of the bacterial suspension was about 150 μL; 80 μL of treated competent cells and 5-20 μg of pPIC9k-GOD-NL-CBM2 plasmids subjected to bgII linearization were added into a 1.5 mL pre-cooled centrifuge tube and evenly mixed. Then, the mixed solution was transferred into a transformation cup (0.2 cm type) that was subjected to ice bath in advance; the transformation cup with the transformation mixed solution was subjected to ice bath for 5 min; an electroporator was arranged according to biorad Pichia pastoris electroporator parameters and electric pulse was started, 1 mL of 1 M sorbitol solution subjected to ice bath was instantly added into the transformation cup after pulse, and then the transformation solution was transferred into a new 1.5 mL centrifuge tube; the transformation solution was cultured by standing for 2 h at 30° C. 100 μL of GS115 transformation solution was sucked and coated onto an MD plate and cultured at 30° C., until transformants appeared. The single clones on the transformed MD plate were subjected to colony PCR verification to ensure the integration of foreign genes.

(5) Induced Expression of GOD-NL-CBM2 Fusion Enzyme Gene

The screened Pichia pastoris recombinants were inoculated in a 5 mL BMGY liquid culture medium and subjected to shaking culture at 30° C. at 250 rpm overnight; 500 μL of overnight cultures were transferred in a 5 mL BMGY liquid culture medium and subjected to shaking culture at 30° C. at 250 rpm overnight until OD600=2-6 (logarithmic growth period, about 16-18 h); the cultures were centrifuged for 5 min at 3000 g, supernatant was discarded, and cells were resuspended with BMMY liquid culture medium until OD600=1.0 (final methanol concentration was 1%), placed in a 500 mL baffled flask, sealed with 8 layers of sterile gauzes and then subjected to shaking culture at 30° C. at 250 rpm; SDS-PAGE detection was performed on the expression of exogenous genes.

(6) Isolation and Purification of GOD-NL-CBM2 Fusion Enzyme

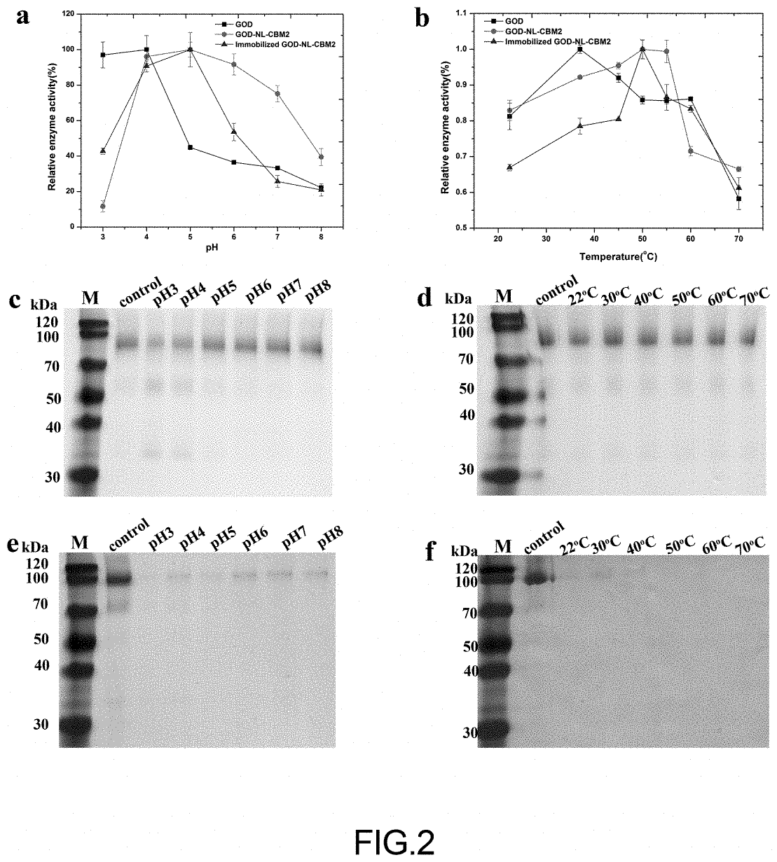

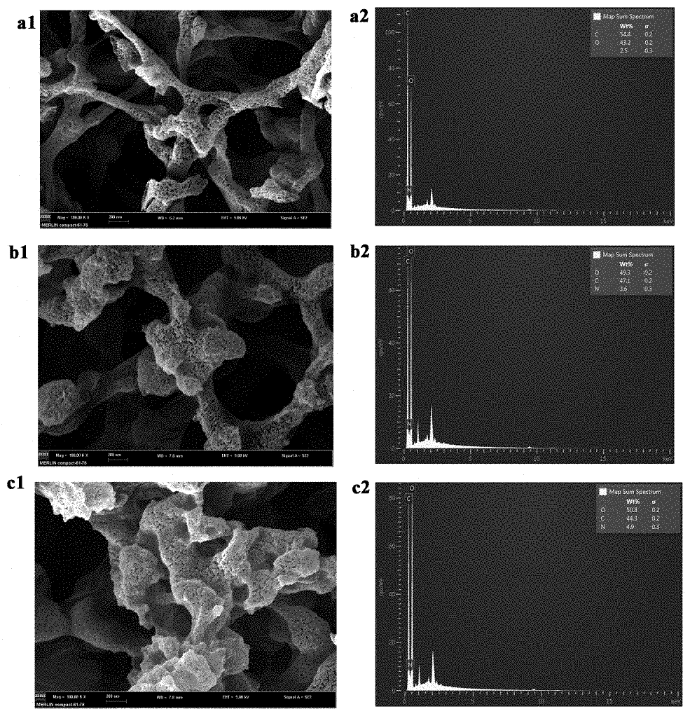

A target protein-expressed crude extract that was detected via SDS-PAGE was mixed with a nickel-containing filler and subjected to rotational binding in a refrigerant at 4° C. for 6 h. Then, heteroproteins were eluted with 5 mM and 10 mM imidazole solutions, a target protein was eluted with a 20 mM imidazole solution and subjected to SDS-PAGE detection. The molecular weight of GOD-NL-CBM2 was obviously higher than that of wild type GOD (FIGS. 1c-1e), indicating that CBM2 had been successfully fused onto a GOD enzyme molecule. In a 3K ultra-filter tube, a protein solution eluted by 20 mM imidazole was ultra-filtered with a pH 5.0 disodium hydrogen phosphate-citrate buffer solution at 4° C. at 4900 rpm, until the pH of the buffer solution flowing down was 5.0, ultra-filtration was stopped, the GOD-NL-CBM2 fusion enzyme solution (1 mg/mL, 200 U/mL) obtained by ultrafiltration was collected, the optimal pH (5.0) and optimal temperature (50° C.) of the fusion enzyme were determined (FIGS. 2a and 2b). The SDS-PAGE detection of the optimal acting temperature and optimal pH results of CBM2 and cellulose in GOD-NL-CBM2 showed that the CBM2 maintained optimal binding activity under the optimal catalytic condition (pH 5.0, 50° C.) (FIGS. 2c-2f).

### Example 2 Morphology and Composition Detection of GOD-NL-CBM2/Cellulose Membrane

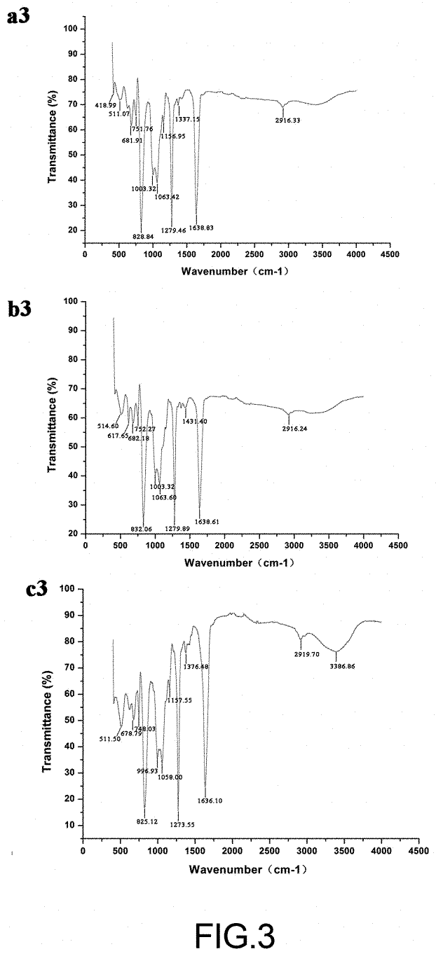

Morphology and composition characterization analysis results of a cellulose membrane (mixed by cellulose nitrate and cellulose acetate) after it did not react with or reacted with GOD using scanning electron microscope (SEM), energy dispersive spectrometer (EDS) and Fourier transform infrared spectroscopy (FTIR) showed that there were different holes with micrometer and nanometer sizes in a three-dimensional structure formed by cellulose nano fibers; meanwhile, nano fibers with a diameter of about 200 nm were in a porous state (FIG. 3a1). After GOD reacted with the cellulose membrane, the diameter of the nano fiber increased, which might be due to expansion effect of a buffer solution, however, there were obvious air holes in the nano fibers, and sizes did not obviously change (FIG. 3b1). In contrast, after GOD-NL-CBM2 interacted with the cellulose membrane, the obvious shape state could be observed, and the surface of the nano fiber seemed to be more smooth (FIG. 3c1).

When the cellulose membrane reacted with GOD, the ratios of C, N and O elements were 54.4%, 2.5% and 43.2%. However, after respectively reacting with GOD and GOD-NL-CBM2, the ratios of C decreased to 47.1% and 44.3%, the ratios of N increased to 3.6% and 4.9%, the ratios of 0 increased to 49.3% and 50.8%. As is known to all, the ratio of N in the protein is higher than that in carbohydrate. Therefore, EDS analysis results provide initial evidence for successful immobilization of GOD-NL-CBM2 on the cellulose membrane.

FTIR detection results showed that the cellulose membrane had characteristic peaks (828 cm⁻¹, 1279 cm⁻¹, 1638 cm⁻¹ and 1050 cm⁻¹) of nitro and cellulose acetate (FIG. 3a3). After the cellulose membrane reacted with GOD, the characteristic peaks did not significant change (FIG. 3b3), however, after GOD-NL-CBM2 reacted with cellulose, there was a significant amide-NH-peak at 3386 cm⁻¹ (FIG. 3c3), which further proved that GOD-NL-CBM2 was immobilized on the cellulose membrane.

### Example 3 Performance Detection of GOD-NL-CBM2 Fusion Enzyme Sensing Element

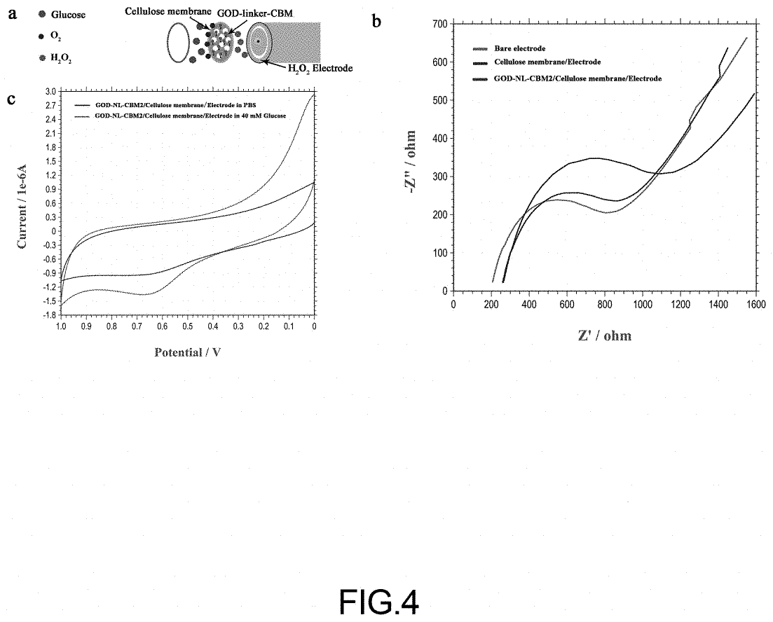

The cellulose membrane was punched into an “O” shape using a puncher and adhered to a rubber ring to prepare an enzyme membrane ring, 20 μL of GOD-NL-CBM2 fusion enzyme solution was directly dropwise added onto the enzyme membrane ring and then dried for 4 h at room temperature, followed by fixing the enzyme membrane ring on an electrode (H₂O₂ electrode) for performance detection (FIG. 4a).

Faraday charge transfer resistor (Rct) detection results of a nude electrode, a cellulose membrane modified electrode and GOD-NL-CBM2/cellulose modified electrode in an electrochemical probe solution showed that compared with the Rct (605.8Ω) of the nude electrode, the Rct of the cellulose membrane modified electrode increased (639.6Ω), and the Rct of the GOD-NL-CBM2 after further acting with the cellulose membrane obviously increased (890.6Ω). Since the conductivity of the cellulose is relatively low, the Rct of the cellulose membrane modified electrode was greater than that of the nude electrode. In addition, the enzyme is usually insulated, increase of Rct after the enzyme is supported indirectly reflects that GOD-NL-CBM2 is successfully immobilized onto the cellulose membrane (FIG. 4b).

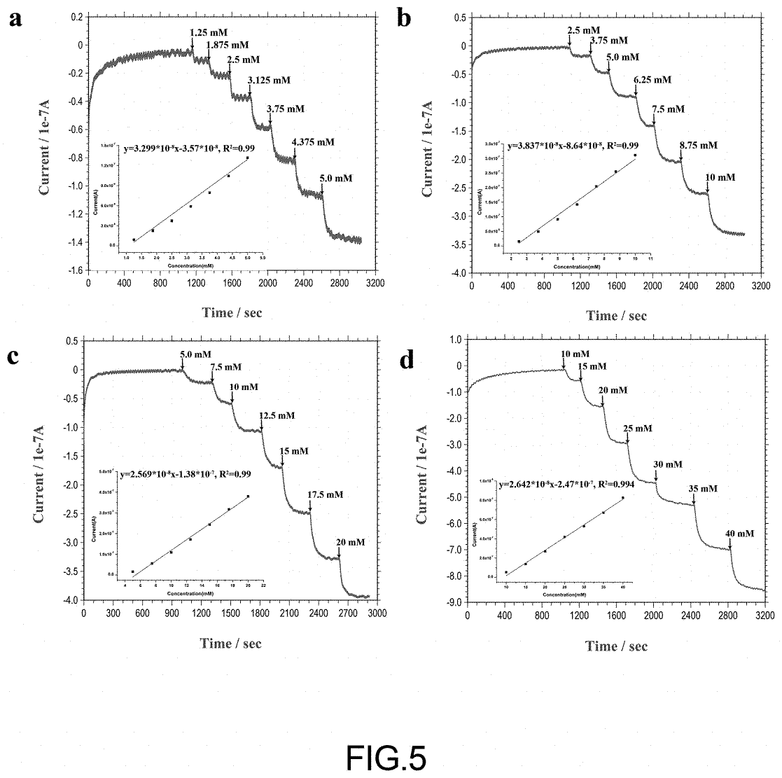

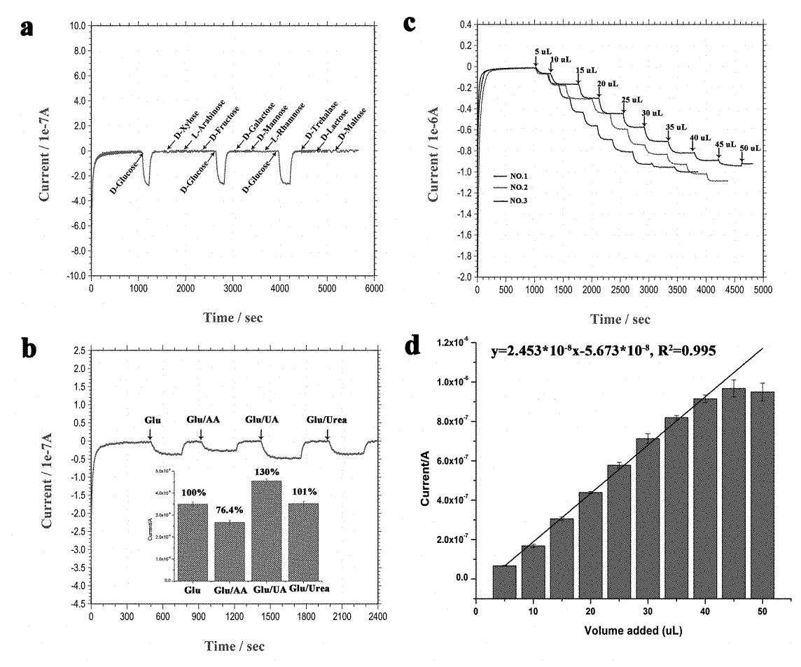

The electrochemical property of an enzyme electrode using a three-electrode system was detected and researched. In a potential range of 0-1.0 V, cyclic voltammetry scanning detection was performed on an electrode with an enzyme membrane in a pH 7.0 PBS buffer solution and a 1 mg/mL glucose solution in turn. The results showed that there are no redox peaks in the PBS buffer solution, and one obvious H₂O₂ peak was detected at +0.6 V in the presence of glucose (FIG. 4c), indicating that the catalytic activity of the GOD enzyme molecule was not changed after CBM2 was fused, with H₂O₂ further generated. Further, by detecting the response of an enzyme membrane on different concentrations of glucose (1.25 mM-5 mM, 2.5 mM-10 mM, 5 mM-20 mM, and 10 mM-40 mM) via chronoamperometry (IT), the obtained results showed that the GOD-NL-CBM2 fusion enzyme sensing element had relatively good electrocatalytic property, and the glucose had a linear detection range of 1.25 mM-40 mM (R2≥0.99) (FIGS. 5a-5b) and a detection limit of 0.475 mM (S/N=3), a sensitivity of 466.7 μA·mol⁻¹·L·cm⁻².

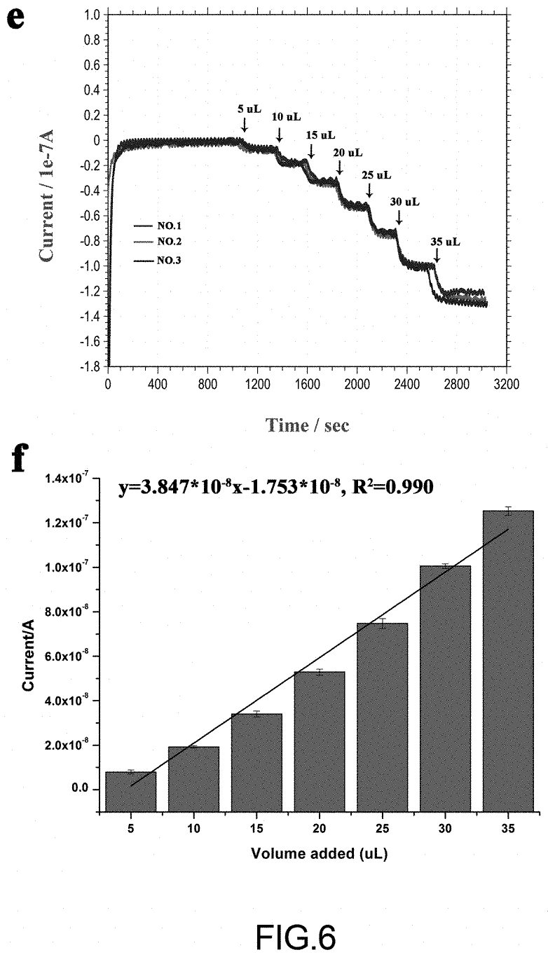

The study results of substrate selectivity and anti-interference ability of GOD-NL-CBM2/cellulose membrane/electrode showed that obvious reaction current signals were not detected after different types of saccharides (D-xylose, L-arabinose, D-fructose, D-neneneba galactose, D-mannose, D-rhamnose, D-trehalose, D-lactose, and D-maltose) reacted with the electrode (FIG. 6a). However, if ascorbic acid (AA, 50 μM) and uric acid (UA, 0.2 mM) were added in glucose, obvious change would be generated (23.6% and 30%), whereas addition of urea did not cause significant change (1%) (FIG. 6b), and the anti-interference ability of the enzyme electrode needs to be further improved.

In addition, the current response signal change of GOD-NL-CBM2/cellulose membrane/electrode was determined everyday for two consecutive months. The results showed that on day 2, the current signal was dropped by 10% compared with initial current signal, whereas in continuous determination, the current signal was slowly dropped, about 80% of initial current signal was remained after 2 months, the service life of the enzyme membrane was >60 days, and above 8000 determinations were continuously performed. Further detection of repeatability of three groups of GOD-NL-CBM2/cellulose membranes/electrodes prepared using the same method showed that when different volumes of 40 mM or 5 mM glucose were added (FIGS. 6c-6f) and the addition volumes were less than 35 three groups of current signal values were similar (RSD<5%), indicating that the GOD-NL-CBM2/cellulose membrane/electrode had relatively high repeatability.

What is claimed is:

1. A fusion protein for a biosensing element, comprising glucose oxidase (GOD) and a carbohydrate-binding module (CBM), and the GOD is linked to the CBM by means of a linker peptide.

2. The fusion protein according to claim 1, wherein the GOD is derived from Aspergillus niger An76.

3. The fusion protein according to claim 1, wherein the CBM is selected from second family CBM.

4. The fusion protein according to claim 1, wherein the linker peptide is selected from (GGGGS)2, (EAAAK)3 or a sequence shown in SEQ ID NO.5.

5. A method for preparing the fusion protein according to claim 1, comprising steps of transforming the coding gene of the fusion protein to Pichia pastoris followed by expressing and purifying.

6. A biosensing element specifically binding to cellulose based on CBM, the biosensing element comprising the fusion protein according to claim 1.

7. The biosensing element according to claim 6, wherein the biosensing element further comprises a cellulose membrane.

8. Use of the fusion protein according to claim 1 in preparing a biosensing element.

9. A method for preparing a biosensing element specifically binding to cellulose based on CBM, comprising a step of contacting the fusion protein according to claim 1 with a cellulose membrane to prepare a fusion protein and cellulose membrane composite.

10. The method according to claim 9, further comprising a step of fixing the composite onto an electrode.

11. The fusion protein according to claim 2, wherein the CBM is selected from second family CBM.

12. A method for preparing the fusion protein according to claim 4, comprising steps of transforming the coding gene of the fusion protein to Pichia pastoris followed by expressing and purifying.


SEQUENCE LISTING (12 sequences)

SEQ ID NO: 1 - protein, 589 aa, synthetic construct
  source 1..589: note = GOD amino acid sequence
LPHYIRSNGIEASLLTDPKEVAGRTVDYIIAGGGLTGLTTAARLTENPDITVLVIESGSYESDRGPIIEDLNAYGDIFGSSVDHAYETVELATNNQTALIRSGNGLGGSTLVNGGTWTRPHKAQVDSWETVFGNEGWNWDSVAAYSLQAERARAPNAKQIAAGHYFNASCHGINGTVHAGPRDTGDDYSPIVKALMSAVEDRGVPTKKDLGCGDPHGVSMFPNTLHEDQVRSDAAREWLLPNYQRPNLQVLTGQYVGKVLLSQNATTPRAVGVEFGTHKGNTHNVYAKHEVLLAAGSAVSPTILEYSGIGMKSILEPLGIDTVVDLPVGLNLQDQTTSTVRSRITSAGAGQGQAAWFATFNETFGDYAEKAHELLNTKLEQWAEEAVARGGFHNTTALLIQYENYRDWIVKDNVAYSELFLDTAGVASFDVWDLLPFTRGYVHILDKDPYLRHFAYDPQYFLNELDLLGQAAATQLARNISNSGAMQTYFAGETIPGDNLAYDADLSAWVEYIPYNFRPNYHGVGTCSMMPKEMGGVVDNAARVYGVQGLRVIDGSIPPTQMSSHVMTVFYAMALKIADAILADYASMQ

SEQ ID NO: 2 - DNA, 1767 bp, synthetic construct
  source 1..1767: mol_type = other DNA; note = GOD nucleic acid sequence
ctcccacactacatcaggagcaatggcatcgaagccagcctcctgactgaccccaaggaggttgccggccgcactgtcgactacatcatcgctggtggaggtctgactggactcaccactgctgcccgtctgacggagaaccccgatatcactgtgcttgtcatcgaaagtggctcctacgagtctgacagaggtcctatcattgaggacctgaacgcttacggtgacatttttggcagcagtgtggaccacgcctacgagactgtcgagctcgccaccaacaatcagactgcgctgatccgctccggaaatggtctcggtggctctaccctcgtcaacggtggcacctggactcgcccccacaaggcacaagttgactcatgggagaccgtcttcggaaatgagggctggaactgggacagcgtggccgcctactccctccaggctgagcgtgctcgcgcaccaaatgccaaacagattgctgctggccactactttaatgcatcctgccatggtatcaatggtactgtccacgccggaccccgcgataccggtgatgactactcccccatcgtcaaggctctcatgagcgctgtcgaagacaggggcgttcccaccaagaaggacttgggatgcggtgacccccatggtgtgtccatgttccccaacaccttgcacgaagaccaagtgcgctctgatgccgctcgcgaatggctcctccccaactaccagcgtcccaacctgcaagtcctcactggacagtatgttggaaaggtcctgctcagccagaacgctaccacacctcgtgccgttggcgtggaattcggcacccacaagggcaacacccacaacgtctacgctaagcacgaggtcctcctggccgctggatccgctgtctctcccaccatcctcgaatattccggtatcggaatgaagtccattctagagcctcttggaattgacaccgtcgttgacctgcccgttggtctcaaccttcaggaccagaccacctctaccgtccgctcacgcattacctccgccggtgccggacagggacaggccgcttggttcgctaccttcaacgagacctttggcgactacgccgaaaaggctcacgagctgctcaacaccaagctggagcagtgggccgaagaggccgtcgcccgtggcggattccacaacaccaccgctttgctcatccagtacgagaactaccgcgactggatcgtcaaggacaatgtcgcatactcggaactcttcctcgacacggccggagtggccagtttcgatgtgtgggatcttctgcccttcactagaggatacgtacacatcctcgacaaggacccctacctccgccatttcgcatacgaccctcagtactttctcaacgagcttgacctgctcggccaggctgccgccactcagctggcccgcaacatctccaactccggtgccatgcaaacttatttcgctggagagactattcccggtgacaacctcgcgtatgatgccgacttgagcgcctgggttgagtatatcccgtacaacttccgccctaactaccatggtgtgggtacttgctccatgatgccgaaggagatgggcggtgttgtcgacaatgctgcccgtgtgtatggtgtgcagggactgcgagtcatcgatggttctattccccctacgcaaatgtcgtcccatgttatgacggtcttttatgccatggccttgaagattgcggatgccatcttggcggattacgcttctatgcag

SEQ ID NO: 3 - protein, 121 aa, synthetic construct
  source 1..121: note = CBM amino acid sequence
PGEPGGPGGPGEPGGPGDGTCAVNYTVVNDWGHGMQGAITVSNTGSSPINNWTLQFSFSGVNISNGWNGEWSQSGSQITVRAPAWNSTLQPGQSVELGFVADKTGNVSPPSQFTLNGATCS

SEQ ID NO: 4 - DNA, 363 bp, synthetic construct
  source 1..363: mol_type = other DNA; note = CBM nucleic acid sequence
ccgggtgagccgggcggtcctggcggtccgggtgaaccgggcggccccggtgacggcacctgcgcggtgaactacaccgtggtcaatgactggggtcacggtatgcagggggcgatcaccgtctccaacaccggatcctcgcccatcaacaactggaccctgcagttcagcttctcgggtgtgaacatctccaacggctggaacggcgagtggagccagagcggctcgcagatcaccgtccgcgctcctgcctggaactccacgctccagccgggccagagcgtggaactgggcttcgttgctgacaagaccggcaacgtctccccgccctcccagttcaccctcaacggagccacctgctcc

SEQ ID NO: 5 - protein, 38 aa, synthetic construct
  source 1..38: note = Linker peptide amino acid sequence
LGGDSSGGGPGEPGGPGGPGEPGGPGGPGEPGGPGDGT

SEQ ID NO: 6 - DNA, 114 bp, synthetic construct
  source 1..114: mol_type = other DNA; note = Linker peptide nucleic acid sequence
ctcggcggcgactcctccggcggcggtccgggtgagccgggcggtcctggcggtccgggtgagccgggcggtcctggcggtccgggtgaaccgggcggccccggtgacggcacc

SEQ ID NO: 7 - protein, 748 aa, synthetic construct
  source 1..748: note = GOD-NL-CBM2 amino acid sequence
LPHYIRSNGIEASLLTDPKEVAGRTVDYIIAGGGLTGLTTAARLTENPDITVLVIESGSYESDRGPIIEDLNAYGDIFGSSVDHAYETVELATNNQTALIRSGNGLGGSTLVNGGTWTRPHKAQVDSWETVFGNEGWNWDSVAAYSLQAERARAPNAKQIAAGHYFNASCHGINGTVHAGPRDTGDDYSPIVKALMSAVEDRGVPTKKDLGCGDPHGVSMFPNTLHEDQVRSDAAREWLLPNYQRPNLQVLTGQYVGKVLLSQNATTPRAVGVEFGTHKGNTHNVYAKHEVLLAAGSAVSPTILEYSGIGMKSILEPLGIDTVVDLPVGLNLQDQTTSTVRSRITSAGAGQGQAAWFATFNETFGDYAEKAHELLNTKLEQWAEEAVARGGFHNTTALLIQYENYRDWIVKDNVAYSELFLDTAGVASFDVWDLLPFTRGYVHILDKDPYLRHFAYDPQYFLNELDLLGQAAATQLARNISNSGAMQTYFAGETIPGDNLAYDADLSAWVEYIPYNFRPNYHGVGTCSMMPKEMGGVVDNAARVYGVQGLRVIDGSIPPTQMSSHVMTVFYAMALKIADAILADYASMQLGGDSSGGGPGEPGGPGGPGEPGGPGGPGEPGGPGDGTPGEPGGPGGPGEPGGPGDGTCAVNYTVVNDWGHGMQGAITVSNTGSSPINNWTLQFSFSGVNISNGWNGEWSQSGSQITVRAPAWNSTLQPGQSVELGFVADKTGNVSPPSQFTLNGATCS

SEQ ID NO: 8 - protein, 2244 aa, synthetic construct
  source 1..2244: note = GOD-NL-CBM2 nucleic acid sequence
CTCCCACACTACATCAGGAGCAATGGCATCGAAGCCAGCCTCCTGACTGACCCCAAGGAGGTTGCCGGCCGCACTGTCGACTACATCATCGCTGGTGGAGGTCTGACTGGACTCACCACTGCTGCCCGTCTGACGGAGAACCCCGATATCACTGTGCTTGTCATCGAAAGTGGCTCCTACGAGTCTGACAGAGGTCCTATCATTGAGGACCTGAACGCTTACGGTGACATTTTTGGCAGCAGTGTGGACCACGCCTACGAGACTGTCGAGCTCGCCACCAACAATCAGACTGCGCTGATCCGCTCCGGAAATGGTCTCGGTGGCTCTACCCTCGTCAACGGTGGCACCTGGACTCGCCCCCACAAGGCACAAGTTGACTCATGGGAGACCGTCTTCGGAAATGAGGGCTGGAACTGGGACAGCGTGGCCGCCTACTCCCTCCAGGCTGAGCGTGCTCGCGCACCAAATGCCAAACAGATTGCTGCTGGCCACTACTTTAATGCATCCTGCCATGGTATCAATGGTACTGTCCACGCCGGACCCCGCGATACCGGTGATGACTACTCCCCCATCGTCAAGGCTCTCATGAGCGCTGTCGAAGACAGGGGCGTTCCCACCAAGAAGGACTTGGGATGCGGTGACCCCCATGGTGTGTCCATGTTCCCCAACACCTTGCACGAAGACCAAGTGCGCTCTGATGCCGCTCGCGAATGGCTCCTCCCCAACTACCAGCGTCCCAACCTGCAAGTCCTCACTGGACAGTATGTTGGAAAGGTCCTGCTCAGCCAGAACGCTACCACACCTCGTGCCGTTGGCGTGGAATTCGGCACCCACAAGGGCAACACCCACAACGTCTACGCTAAGCACGAGGTCCTCCTGGCCGCTGGATCCGCTGTCTCTCCCACCATCCTCGAATATTCCGGTATCGGAATGAAGTCCATTCTAGAGCCTCTTGGAATTGACACCGTCGTTGACCTGCCCGTTGGTCTCAACCTTCAGGACCAGACCACCTCTACCGTCCGCTCACGCATTACCTCCGCCGGTGCCGGACAGGGACAGGCCGCTTGGTTCGCTACCTTCAACGAGACCTTTGGCGACTACGCCGAAAAGGCTCACGAGCTGCTCAACACCAAGCTGGAGCAGTGGGCCGAAGAGGCCGTCGCCCGTGGCGGATTCCACAACACCACCGCTTTGCTCATCCAGTACGAGAACTACCGCGACTGGATCGTCAAGGACAATGTCGCATACTCGGAACTCTTCCTCGACACGGCCGGAGTGGCCAGTTTCGATGTGTGGGATCTTCTGCCCTTCACTAGAGGATACGTACACATCCTCGACAAGGACCCCTACCTCCGCCATTTCGCATACGACCCTCAGTACTTTCTCAACGAGCTTGACCTGCTCGGCCAGGCTGCCGCCACTCAGCTGGCCCGCAACATCTCCAACTCCGGTGCCATGCAAACTTATTTCGCTGGAGAGACTATTCCCGGTGACAACCTCGCGTATGATGCCGACTTGAGCGCCTGGGTTGAGTATATCCCGTACAACTTCCGCCCTAACTACCATGGTGTGGGTACTTGCTCCATGATGCCGAAGGAGATGGGCGGTGTTGTCGACAATGCTGCCCGTGTGTATGGTGTGCAGGGACTGCGAGTCATCGATGGTTCTATTCCCCCTACGCAAATGTCGTCCCATGTTATGACGGTCTTTTATGCCATGGCCTTGAAGATTGCGGATGCCATCTTGGCGGATTACGCTTCTATGCAGCTCGGCGGCGACTCCTCCGGCGGCGGTCCGGGTGAGCCGGGCGGTCCTGGCGGTCCGGGTGAGCCGGGCGGTCCTGGCGGTCCGGGTGAACCGGTGACGGCACCCCGGGTGAGCCGGGCGGTCCTGGCGGTCCGGGTGAACCGGGCGGCCCCGGCGGCCCCGGGTGACGGCACCTGCGCGGTGAACTACACCGTGGTCAATGACTGGGGTCACGGTATGCAGGGGGCGATCACCGTCTCCAACACCGGATCCTCGCCCATCAACAACTGGACCCTGCAGTTCAGCTTCTCGGGTGTGAACATCTCCAACGGCTGGAACGGCGAGTGGAGCCAGAGCGGCTCGCAGATCACCGTCCGCGCTCCTGCCTGGAACTCCACGCTCCAGCCGGGCCAGAGCGTGGAACTGGGCTTCGTTGCTGACAAGACCGGCAACGTCTCCCCGCCCTCCCAGTTCACCCTCAACGGAGCCACCTGCTCC

SEQ ID NO: 9 - DNA, 43 bp, synthetic construct
  source 1..43: mol_type = other DNA; note = PCR amplification P1
gctgaagcttacgtagaattcctcccacactacatcaggagca

SEQ ID NO: 10 - DNA, 22 bp, synthetic construct
  source 1..22: mol_type = other DNA; note = PCR amplification P2
gaggtggtggtggtggtggtgc

SEQ ID NO: 11 - DNA, 35 bp, synthetic construct
  source 1..35: mol_type = other DNA; note = PCR amplification P3
accaccaccaccaccacctcggcggcgactcctcc

SEQ ID NO: 12 - DNA, 43 bp, synthetic construct
  source 1..43: mol_type = other DNA; note = PCR amplification P4
aaggcgaattaattcgcggccgctcagtggtggtggtggtggt